(19) United States
(12) Patent Application Publication
(10) Pub. No.: US 2012/0264830 A1
(43) Pub. Date: Oct. 18, 2012

(54) CHONDROITIN SULFATE-POLYCAPROLACTONE COPOLYMER, METHOD FOR PREPARING THE SAME AND APPLICATION THEREOF

(75) Inventors: Li-Fang Wang, Kaohsiung City (TW); Yu-Sheng LIU, Minsyong Township (TW)

(73) Assignee: KAOHSIUNG MEDICAL UNIVERSITY, Kaohsiung (TW)

(21) Appl. No.: 13/326,835

(22) Filed: Dec. 15, 2011

(30) Foreign Application Priority Data
Apr. 15, 2011 (TW) 100113184

(51) Int. Cl. A61K 47/34; C08G 63/91; C08G 63/08; B82Y 5/00; B82Y 40/00

(52) U.S. Cl. 514/772.1; 525/54.2; 977/788; 977/906

(57) ABSTRACT

A method for preparing a chondroitin sulfate-polycaprolactone copolymer includes subjecting a chondroitin sulfate component and a polycaprolactone polymer to an atom transfer radical polymerization reaction in the presence of a catalyst.

### BACKGROUND OF THE INVENTION

1. Field of the Invention

The present invention relates to a method for preparing a polycaprolactone copolymer, more particularly to a method for preparing a chondroitin sulfate-polycaprolactone copolymer. The present invention also relates a chondroitin sulfate-polycaprolactone copolymer, a nano-micelle carrier made from the chondroitin sulfate-polycaprolactone copolymer, and a medical composition comprising the chondroitin sulfate-polycaprolactone copolymer.

2. Description of the Related Art

Cancer, a disease of unregulated cell growth, is caused by DNA mutation and transforms normal cells into cancer cells. These abnormal cells expand locally due to their high invasiveness and spread systemically by metastasis. At present, methods for treating cancer generally include surgical excision, radiation treatment, chemical therapy, and the like. In general, although the surgical excision may prolong the life of a patient, healing ability is weak. In addition, the radiation treatment and chemical therapy may cause damage to normal cells. Therefore, selecting a drug carrier that is safe and stable, with the properties of high selectivity to tumor cell/organ is the future of cancer medical therapy.

Since polycaprolactone polymers have substantial biocompatibility, most of the sutures, bone pegs and cell regeneration templates use polycaprolactone as a material thereof. Therefore, there have been many studies about taking the polycaprolactone compound as a drug carrier. Although the polycaprolactone may be metabolized into carbon dioxide and water through a citric cycle in vivo, the biodegradation speed of the polycaprolactone polymer is relatively slow. Therefore, further modification of the polycaprolactone polymer is required. It is known that polyethylene glycol-polycaprolactone (referred to as PEG-PLC) carrier and dextran-polycaprolactone carrier (referred to as DEX-PLC) have been widely studied and developed.

The PEG in the PEG-PLC carrier is not biodegradable. Further, since the DEX-PLC carrier is susceptible to be identified as a foreign object by human immune system, it is difficult to circulate in the blood for a long time. In addition, each of the aforesaid two carriers has a high critical micelle concentration that leads to difficult self-assembly and low drug carriability. Therefore, they are not suitable to serve as drug carriers. A polycaprolactone graft-chondroitin sulfate and a synthesis method thereof are disclosed in Biomacromolecules 2008, 9, 2447-2457. The polycaprolactone graft-chondroitin sulfate is represented by the following formula:

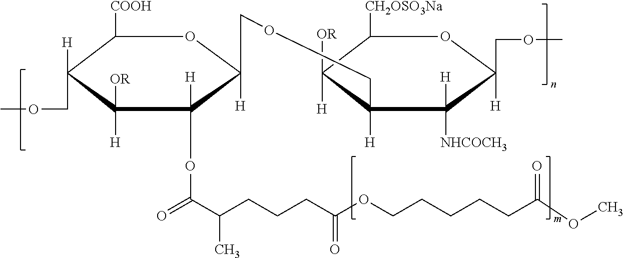

wherein R is H or

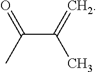

The polycaprolactone graft-chondroitin sulfate is obtained by reacting polycaprolactone polymer with a chondroitin sulfate modified with a double bond compound. However, the synthesis method is carried out by a conventional radical polymerization reaction initiated by azobis-isobutyronitrile (AIBN) that lacks specificity and is liable to produce by-products. A plurality of steps is required to remove the by-products. Meanwhile, each of chondroitin sulfate and polycaprolactone polymers, both of which are modified with double bond compounds, is liable to undergo a self-cross-linking reaction or self-polymerization, thereby resulting in a low yield (45%˜55%) for polycaprolactone graft-chondroitin sulfate. In addition, the critical micelle concentration of the resultant polycaprolactone graft-chondroitin sulfate is high (3.17×10⁻³ mg/mL), and thus is difficult to exist stably in the circulating blood.

### SUMMARY OF THE INVENTION

Therefore, a first object of the present invention is to provide a method for preparing a chondroitin sulfate-polycaprolactone copolymer that has a high yield, that simplifies the purification steps, and that can effectively control the grafting ratio.

A second object of the present invention is to provide a chondroitin sulfate-polycaprolactone copolymer that has an improved biocompatibility, critical micelle concentration, and cancer cell targeting ability.

A third object of the present invention is to provide a nano-micelle carrier that has a high active ingredient carriability and enclosed percentage.

A fourth object of the present invention is to provide a medical composition that improves active ingredient releasability.

According to a first aspect of the present invention, there is provided a method for preparing a chondroitin sulfate-polycaprolactone copolymer, comprising subjecting a chondroitin sulfate component and a polycaprolactone polymer to an atom transfer radical polymerization reaction in the presence of a catalyst.

According to a second aspect of the present invention, there is provided a chondroitin sulfate-polycaprolactone copolymer, which prepared according to the method of the first aspect.

According to a third aspect of the present invention, there is provided a nano-micelle carrier obtained by subjecting the chondroitin sulfate-polycaprolactone copolymer according to the second aspect to a dialysis treatment.

According to a fourth aspect of the present invention, there is provided a medical composition comprising the chondroitin sulfate-polycaprolactone copolymer according to the second aspect and an active ingredient.

According to a fifth aspect of the present invention, there is provided a medical composition comprising the nano-micelle carrier according to the third aspect and an active ingredient.

### BRIEF DESCRIPTION OF THE DRAWINGS

Other features and advantages of the present invention will become apparent in the following detailed description of the preferred embodiments with reference to the accompanying drawings, of which:

FIG. 1 is a NMR graph illustrating the structure analysis of a modified chondroitin sulfate that is modified with a double-bond compound and that is used in a preferred embodiment of this invention;

FIG. 2 is a NMR graph illustrating the structure analysis of a polycaprolactone polymer used in a preferred embodiment of this invention;

FIG. 3 is a NMR graph illustrating the structure analysis of the preferred embodiment of a chondroitin sulfate-polycaprolactone copolymer according to the present invention;

FIG. 4 is a graph illustrating the critical micelle concentration of the preferred embodiment of a chondroitin sulfate-polycaprolactone copolymer according to the present invention;

FIG. 5 is a bar diagram illustrating the cytotoxicity of the preferred embodiment of a chondroitin sulfate-polycaprolactone copolymer according to the present invention with respect to CRL-5802 cells;

FIG. 6 is a bar diagram illustrating the killing ability of the preferred embodiment of a chondroitin sulfate-polycaprolactone copolymer according to the present invention against the CRL-5802 cells;

FIG. 7 is an image illustrating the internalization of the preferred embodiment of a chondroitin sulfate-polycaprolactone copolymer according to the present invention in the CRL-5802 cells; and

FIG. 8 is a graph illustrating the camptothecin releasability of the preferred embodiment of a chondroitin sulfate-polycaprolactone copolymer according to the present invention.

### DETAILED DESCRIPTION OF THE PREFERRED EMBODIMENTS

The method for preparing a chondroitin sulfate-polycaprolactone copolymer according to the present invention comprises subjecting a chondroitin sulfate component and a polycaprolactone polymer to an atom transfer radical polymerization reaction in the presence of a catalyst to obtain the chondroitin sulfate-polycaprolactone copolymer. The chondroitin sulfate component includes a modified chondroitin sulfate modified with a double-bond compound. The polycaprolactone polymer is represented by the following formula (I) and has a weight average molecular weight ranging from 2000 to 10000,

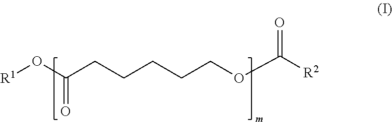

wherein, in formula (I), R¹ is a C₁-C₈ straight or branched alkyl group, an aromatic group, or

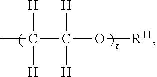

R¹¹ being H or methyl, t being an integer ranging from 45 to 225;

R² is

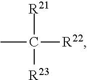

R²¹, R²², and R²³ being independently H, methyl, or halogen atom, with the proviso that at least one of R²¹, R²², and R²³ is halogen atom, and

m is an integer ranging from 18 to 88.

In the method of the present invention, the catalyst may initiate the departure of halogen so as to produce a free radical in the polycaprolactone polymer. The polycaprolactone polymer containing the free radical is then reacted with a double bond functional group contained in the chondroitin sulfate component by an atom transfer radical polymerization (ATRP) such that the polycaprolactone polymer can be more specifically grafted to the chondroitin sulfate component as compared to the conventional method initiated by the azobis-isobutyronitrile initiator. Such an ATRP method may improve the reaction selectivity, and may increase the yield to above 70%. In addition, it may prevent the self cross-linking reaction of a modified chondroitin sulfate modified with a double-bond compound contained in the chondroitin sulfate component and may reduce the generation of by-products so as to avoid complicated purification steps, thereby reducing the manufacturing cost. When the chondroitin sulfate-polycaprolactone copolymer made from the conventional method initiated by azobis-isobutyronitrile (AIBN), each of chondroitin sulfate and polycaprolactone should be modified with a double bond compound, which is liable to undergo a self-cross-linking reaction or self-polymerization, thereby resulting in a low yield and difficulty in purification. Further, because the polycaprolactone polymer is a hydrophobic compound, and the chondroitin sulfate component is a hydrophilic compound, a special consideration is required to select a solvent used in the reaction of the polycaprolactone polymer and the chondroitin sulfate component. However, the method of the present invention can be carried out in a non-homogeneous condition. Even when a co-solvent may not be found and thus the polycaprolactone polymer and the chondroitin sulfate component may not be completely dissolved, the reaction can still be performed effectively.

The method of the present invention may be used to graft polycaprolactone polymer having different molecular weights. Therefore, the method of the present invention is greatly superior to the conventional method that can only be used to graft the polycaprolactone polymer having a molecular weight of about 2,000.

Preferably, the reaction temperature of the atom transfer radical polymerization reaction ranges from 55° C. to 65° C. Preferably, the reaction time of the atom transfer radical polymerization reaction ranges from 1 hour to 2 hours. Preferably, the weight ratio of the chondroitin sulfate component to the polycaprolactone polymer ranges from 0.1 to 0.9.

Preferably, the catalyst may be selected from copper bromide (CuBr), copper chloride (CuCl), and a combination thereof.

Preferably, a solvent may be added to the atom transfer radical polymerization reaction. The solvent maybe selected from dimethyl sulfoxide (DMSO), toluene, 1,4-dioxane, xylene, anisole, dimethyl formamide (DMF), water, methanol, acetonitrile (ACN), chloroform, or combinations thereof.

The chondroitin sulfate component comprises at least a modified chondroitin sulfate obtained by subjecting chondroitin sulfate and a double-bond compound to a polymerization reaction. The chondroitin sulfate is selected from chondroitin sulfate A, chondroitin sulfate C, chondroitin sulfate E, and combinations thereof. The double-bond compound is selected from acrylic acid, acrylic anhydride, acryol chloride, methacrylic acid, methacrylic anhydride, methacryol chloride and methyl methacrylate. The reason for selection of the chondroitin sulfate serving as a part of a carrier according to this invention is that the chondroitin sulfate is an important ingredient in connective tissues and is a polysaccharide that may not be identified as a foreign object by human immune system. In addition, in the present invention, the chondroitin sulfate is modified to form the modified chondroitin sulfate such that the polycaprolactone polymer can be effectively grafted with the chondroitin sulfate so as to increase the grafting ratio and improve the reaction selectivity. The process for preparing the modified chondroitin sulfate comprises the step of stirring and mixing uniformly the chondroitin sulfate and the double-bond compound under alkali environment. Preferably, the molar ratio of the chondroitin sulfate to the double-bond compound ranges from 0.05 to 0.8. Preferably, the reaction temperature ranges from 25° C. to 30° C. Preferably, the reaction time ranges from 18 hours to 36 hours.

The polycaprolactone polymer is obtained by subjecting the caprolactone to a ring opening polymerization that is well known in the art and is not described in detail herein below. The polycaprolactone polymer is represented by the following formula (I) and has a weight average molecular weight ranging from 2000 to 10000,

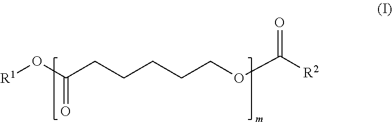

wherein, in formula (I), R¹ is a C₁-C₈ straight or branched alkyl group, an aromatic group, or

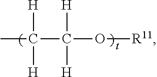

R¹¹ being H or methyl, t being an integer ranging from 45 to 225; R² is

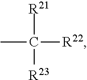

R²¹, R²², and R²³ being independently H, methyl, or halogen atom, with the proviso that at least one of R²¹, R²², and R²³ is halogen atom, and m is an integer ranging from 18 to 88.

Preferably, in formula (I), R¹ is a C₁-C₈ straight or branched alkyl group, a benzyl group,

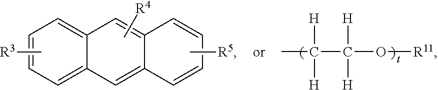

wherein R¹¹ is H or methyl, R³, R⁴, and R⁵ are independently H or an alkylene group, with the proviso that at least one of R³, R⁴, and R⁵ is an alkylene group. In an example of the present invention, R¹ is a benzyl group.

Preferably, in formula (I), R² is

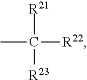

R²¹, R²², and R²³ being independently H, methyl, Cl, or Br, with the proviso that at least one of R²¹, R²², and R²³ is Cl or Br.

More preferably, in formula (I), R² is

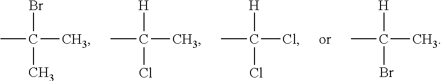

In an example of the present invention, R² is

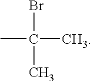

The chondroitin sulfate-polycaprolactone copolymer of this invention has biocompatibility and biodegradability. When the chondroitin sulfate-polycaprolactone copolymer of this invention is used to enclose an active ingredient (for example, anti-cancer drug or anti-oxidant) so as to form a medical composition, the active ingredient may not be released until the medical composition reaches target cells, thereby reducing the probability of the active ingredient being degraded during the process of delivery. When the medical composition reaches the target cells, the active ingredient maybe released at a predetermined rate so as to achieve therapeutic effect. Moreover, it is known that the low solubility problem exists commonly in most of the anti-cancer drugs that serve as the active ingredients. Because the chondroitin sulfate-polycaprolactone copolymer of the present invention has a hydrophilic chondroitin sulfate, and can be self-assembled in water to form micelles, when the same is used to enclose the anti-cancer drug, the low solubility problem of the anti-cancer drug can be overcome, thereby facilitating delivery of the active ingredient to the target cells. Preferably, based on 100% of the total weight of the chondroitin sulfate-polycaprolactone copolymer, the content of the polycaprolactone group ranges from about 20 wt % to 90 wt %. Besides, the chondroitin sulfate-polycaprolactone copolymer may be reacted with biomolecules (for example, folic acid, peptide, fluorescence molecules, etc.) with the use of the acidic groups on the chondroitin sulfate so as to prepare a multi-functional target drug delivery carrier.

A nano-micelle carrier of the present invention is obtained by subjecting the chondroitin sulfate-polycaprolactone copolymer described above to a dialysis treatment.

Preferably, the dialysis treatment involves the step of self-assembling the chondroitin sulfate-polycaprolactone copolymer to form micelles. A relatively low concentration of the chondroitin sulfate-polycaprolactone copolymer may not form micelles in a water solution. When the concentration of the chondroitin sulfate-polycaprolactone copolymer is larger than the critical micelle concentration (CMC), due to the intermolecular force of the hydrophobicity, hydrogen bond and the like, the polycaprolactone components may aggregate together to form a hydrophobic core while the chondroitin sulfate components form a hydrophilic shell, thereby increasing the stability of the micelle structure. In the nano-medical field, micelle carriers are very important and are expected to have the following properties: small particle size, high carriability and enclosed percentage for an active ingredient, an excellent structural stability, and low turnover rate in vivo. With the aforesaid properties, the micelle carrier of the present invention can effectively deliver the active ingredient to the target cells. In addition, it is further expected that the particle size of the micelle be controlled at a nanometer range in order to have an improved selective permeation of the vascular wall.

Preferably, the critical micelle concentration of the chondroitin sulfate-polycaprolactone copolymer ranges from 1.3×10⁻³ mg/mL to 2.2×10⁻³ mg/mL, which is lower than that of the polycaprolactone graft-chondroitin sulfate disclosed in Biomacromolecules.

A medical composition of the present invention comprises the chondroitin sulfate-polycaprolactone copolymer described above and an active ingredient.

The chondroitin sulfate-polycaprolactone copolymer is a hydrophilic-hydrophobic polymer so that, when the chondroitin sulfate-polycaprolactone copolymer is used to enclose a hydrophobic active ingredient, the hydrophobic polycaprolactone core exhibits a higher interaction with the hydrophobic active ingredient and can be us ed to enclose the hydrophobic active ingredient.

The hydrophilic chondroitin sulfate shell facilitates effective delivery of the active ingredient to the target cells.

The abovementioned active ingredient refers to a substance that can be used to diagnose, treat, mitigate, or prevent human disease or that can be used to affect the human body structure or physiological function. Preferably, the active ingredient is selected from camptothecin (CPT), doxorubicin (DOX), topotecan, cyclosproine A, epriubicin, rapamycin, vitamin A, vitamin D, vitamin E and vitamin K, paclitaxel, and combinations thereof. In an example of the present invention, the active ingredient is camptothecin and doxorubicin. The weight ratio of the active ingredient to the chondroitin sulfate-polycaprolactone copolymer ranges from 0.01 to 0.2. A medical composition of the present invention comprises the nano-micelle carrier described above and an active ingredient. The merit of the nano-micelle carrier is that the active ingredient is protected from the destruction of the human immune system, resulting in increased stability of the active ingredient in the blood, thereby prolonging the retention time of the active ingredient in the blood. Preferably, the weight ratio of the active ingredient to the nano-micelle carrier ranges from 0.01 to 0.2.

The enclosed percentage and the carriability of the medical composition of the present invention varies depending on the types of the active ingredients. Preferably, when camptothecin is used as the active ingredient, the enclosed percentage of the medical composition of the present invention ranges from 30% to 50% and the carriability ranges from 3% to 5%. Preferably, when doxorubicin is used as the active ingredient, the enclosed percentage of the medical composition of the present invention ranges from 40% to 70% and the carriability ranges from 4% to 7%.

### EXAMPLE

[Preparation of a Modified Chondroitin Sulfate Modified with a Double-Bond Compound]

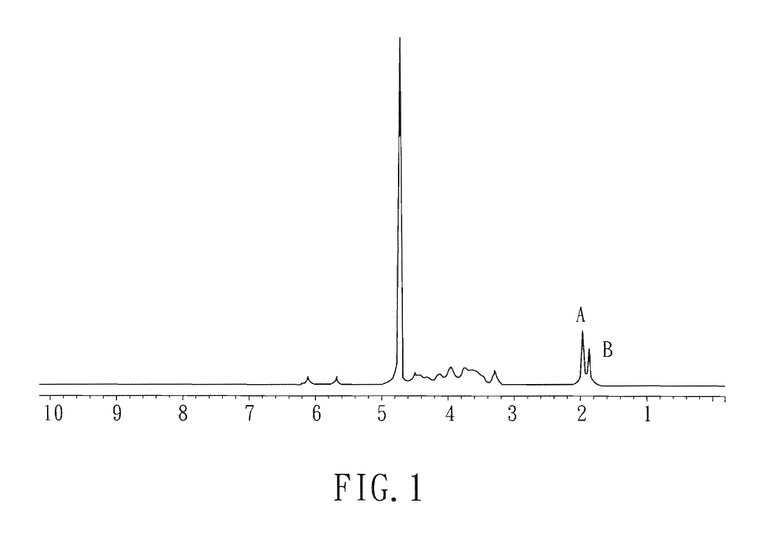

1 g of the chondroitin sulfate commercially available from Tooku Miyagi (Mw=58,000 Da) was added into 50 mL of water with stirring and was dissolved uniformly, followed by adding dropwise 12 mL of methacrylic anhydride commercially available from Lancaster (Mw=90.51 g/mol) and mixing uniformly so as to form a mixture. Subsequently, 18 mL of 5N sodium hydroxide solution was added dropwise into the mixture, followed by reaction at room temperature for two days so as to form a reaction solution. The reaction solution was then placed in a refrigerator at 4° C. for 24 hours. Thereafter, the reaction solution was added dropwise into 50 times volume of ethanol, followed by centrifugation at 6,500 rpm for 5 minutes. After the supernatant was removed, the precipitate was washed with 50 mL of ethanol three times to remove the un-reacted methacrylic anhydride and the reacted chondroitin sulfate component. The washed precipitate as white powder was collected and a modified chondroitin sulfate was obtained. Subsequently, the modified chondroitin sulfate was dried in a vacuum oven. The structural analysis of the dried modified chondroitin sulfate is shown in FIG. 1.

Since, in the chondroitin sulfate structure, there are three OH groups that can be used to graft the methacrylic anhydride, it is defined that the maximum bonding extent of the methacrylic anhydride on the chondroitin sulfate is 300. The symbol “A” in FIG. 1 represents three H's on the amide group of the chondroitin sulfate and the symbol “B” in FIG. 1 represents three H's on the methyl group of the methacrylic anhydride. The grafting ratio of the modified chondroitin sulfate may be obtained by dividing the integrated area of B by the integrated area of A. The grafting ratio of the modified chondroitin sulfate of the present invention is 70%.

[Preparation of Polycaprolactone Polymer]

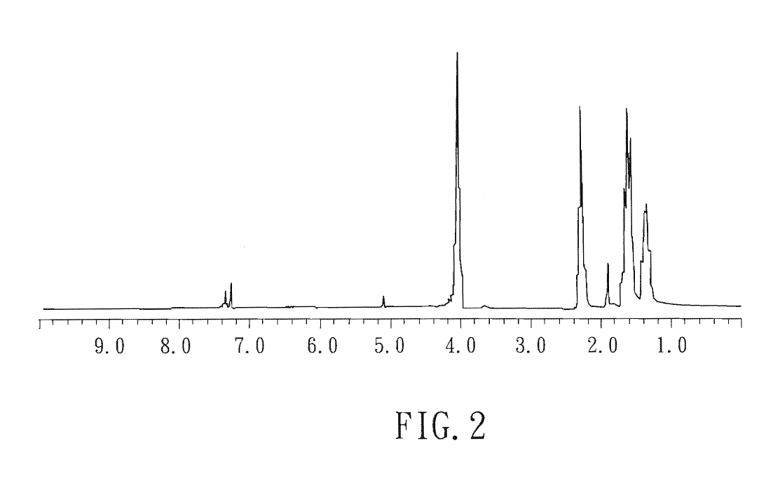

0.24 mL of phenylmethyl alcohol and 14 g of ε-caprolactone were mixed at 100° C. to conduct a ring-opening reaction for 2 hours to obtain a product (Molecular weight: 6,000). The product was placed in a reaction flask and a deoxygenation process was conducted for 30 minutes. Subsequently, 50 mL of dichloromethane was added to dissolve the product followed by addition of triethylamine (TEA) and 2-bromo-2-methylpropionyl bromide to form a mixture. The mixture was immersed in an ice bath for one day to obtain a polycaprolactone polymer. The structural analysis of the polycaprolactone polymer is shown in FIG. 2.

[Preparation of Chondroitin Sulfate-Polycaprolactone Copolymer]

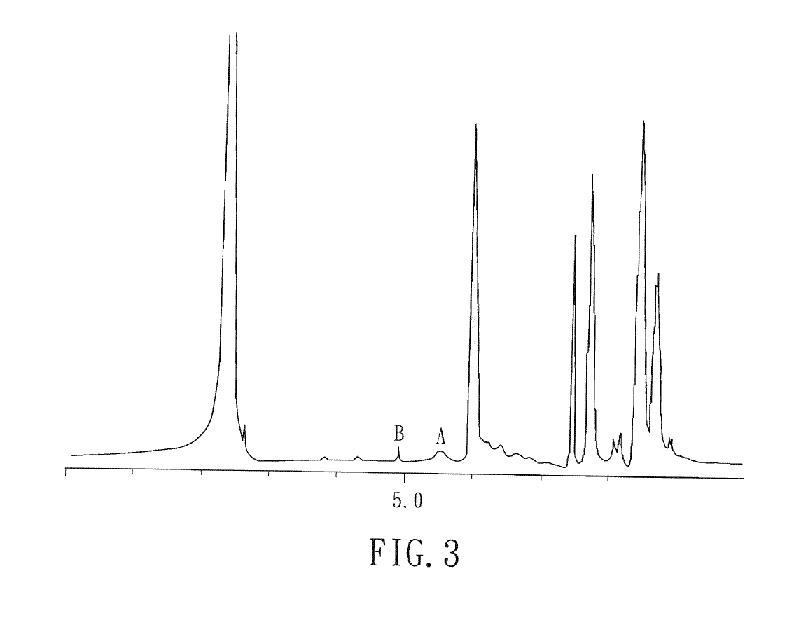

50 mg of the modified chondroitin sulfate was dissolved in water to obtain a first reactant. 100 mg of the polycaprolactone polymer was dissolved in dimethyl sulfoxide (DMSO) to obtain a second reactant. The first reactant and the second reactant were mixed, frozen and deoxygenated three times. Subsequently, copper bromide and bipyridine were added into the resultant mixture, followed by reaction in an oil bath for 2 hours to obtain a chondroitin sulfate-polycaprolactone copolymer having a yield of 70%. The structural analysis of the chondroitin sulfate-polycaprolactone copolymer is shown in FIG. 3. The grafting ratio and the content of polycaprolactone in the chondroitin sulfate-polycaprolactone copolymer can be calculated from FIG. 3.

Calculation of the grafting ratio: dividing the integrated area (B) of two H's on the phenyl group of the polycaprolactone at 5.1 ppm by the integrated area (A) of two H's in the repeating unit of the chondroitin sulfate at 4.4 ppm to calculate the molar content of the polycaprolactone grafted on each of the repeating units of the chondroitin sulfate.

Calculation of the content of the polycaprolactone: (grafting ratio×molecular weight of the polycaprolactone polymer)/[(molecular weight of the repeating unit of the chondroitin sulfate×(100−grafting ratio))+(grafting ratio×molecular weight of the polycaprolactone polymer)], in which the molecular weight of the repeating unit of the chondroitin sulfate of the preferred embodiment of the present invention is 528 g/mole.

[Preparation of Nano-Micelle Carrier]

10 mg of the chondroitin sulfate-polycaprolactone copolymer was dissolved in 5 mL of DMSO containing 4 μL of trifluoroacetic acid (TFA) at 60° C. The mixture was cooled to room temperature and subjected to a dialysis treatment in de-ionized water using a dialysis membrane (Merck, molecular weight cut-off: 6000 to 8000). The de-ionized water was changed every three hours for three days. A nano-micelle carrier was thus obtained.

[Preparation of Medical Composition]

10 mg of the chondroitin sulfate-polycaprolactone copolymer was dissolved in 5 mL of DMSO containing 4 μL of trifluoroacetic acid (TFA) at 60° C. to form a first solution. 1 mg of camptothecin used as an active ingredient was dissolved in DMSO to form a second solution. The first and second solutions were mixed and subjected to a dialysis treatment in 2 L of pure water using a dialysis membrane (Merck, molecular weight cut-off: 6000 to 8000). The pure water was changed every three hours for two days. After the dialysis treatment was finished, a liquid in the dialysis membrane was lyophilized. The lyophilized product was dissolved in water and filtered using a filter paper to remove the unenclosed camptothecin that was not dissolved in water. The filtrate was collected and lyophilized to obtain a medical composition. The carriability of the camptothecin of the medical composition is calculated based on the following equation (1) and is about 4%.

$$Carriability = \frac{{W}_{c}}{{W}_{m}} \times 100 \% \qquad ( 1 )$$

W_{c}: the weight of the enclosed camptothecin in the medical composition
Wₘ: the weight of the medical composition

### <Evaluation>

1. Determination of Critical Micelle Concentration (CMC)

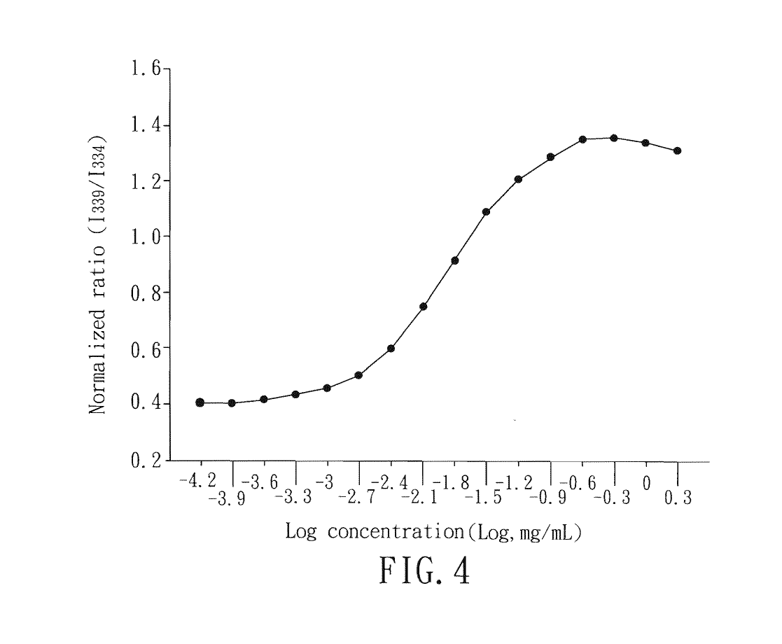

The resultant chondroitin sulfate-polycaprolactone copolymer and de-ionized water were mixed to prepare a stock solution having a concentration of 2 mg/mL. The stock solution was then diluted to obtain 15 diluted solutions with different concentrations (1 mg/mL, 0.5 mg/mL, 0.25 mg/mL, 0.125 mg/mL, 0.0625 mg/mL, 0.03125 mg/mL, 0.0156 mg/mL, 0.0078 mg/mL, 0.004 mg/mL, 0.002 mg/mL, 0.001 mg/mL, 0.0005 mg/mL, 0.00025 mg/mL, 0.0001 mg/mL, 0.00005 mg/mL). 6.0×10⁻⁷ M of pyrene was added to the stock solution and 15 diluted solutions. To determine CMC, fluorescence measurement was performed using a fluorescence spectrophotometer. The excitation spectra were recorded from 300 to 360 nm with an emission wavelength of 390 nm. Normalized intensity for the 339 nm and 334 nm peaks were measured, and the normalized ratio (I₃₃₉/I₃₃₄) is plotted against the copolymer log concentration (see FIG. 4). From FIG. 4, the CMC value of the chondroitin sulfate-polycaprolactone copolymer calculated from the intersection of two tangent plots of I₃₃₉/I₃₃₆ against the copolymer log concentration is 1.3×10⁻³ mg/mL, which is lower than that of the polycaprolactone graft-chondroitin sulfate disclosed in Biomacromolecules set forth in the section of “2. Description of the Related Art”. The result indicates that the chondroitin sulfate-polycaprolactone copolymer of this invention is different from that of the prior art and can be formed into micelles at a lower concentration, i.e., has an improved self-assembly ability.

2. Cytotoxicity Test Against Lung Cancer Cell CRL-5802

(a) Cytotoxicity test of chondroitin sulfate-polycaprolactone copolymer against lung cancer cell CRL-5802:

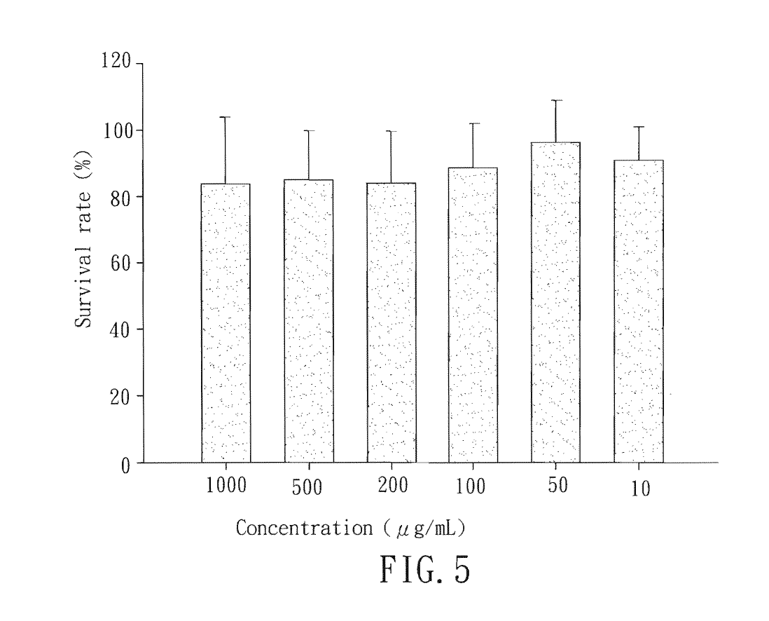

5000 cells/well of CRL-5802 were placed in a 96-well plate containing 100 μL of Dulbecco's Modified Eagle's Medium (DMEM, manufacturer: Invitrogen), followed by cultivation for 24 hours at 37° C. and 5% CO₂. The cell culture medium was periodically replaced during the cultivation period. Thereafter, the aforesaid stock solution that was described in the section of “1. Determination of critical micelle concentration” under “Evaluation” and that has 2 mg/mL of the chondroitin sulfate-polycaprolactone copolymer was diluted with DMEM to obtain five diluted solutions with different concentrations (1000 μg/mL, 400 μg/mL, 200 μg/mL, 100 μg/mL and 20 μg/mL). Subsequently, the stock solution and the 5 diluted solutions were added into the respective wells containing cells so that the final concentrations of the chondroitin sulfate-polycaprolactone copolymer were adjusted respectively to 1000 μg/mL, 500 μg/mL, 200 μg/mL, 100 μg/mL, 50 μg/mL, and 10 μg/mL (experimental group).The cells in the control group were cultured with DMEM without addition of the chondroitin sulfate-polycaprolactone copolymer. The cells were further cultivated for 24 hours. Thereafter, 50 mL of thiazolyl blue tetrazolium bromide (MTT) was added into each of the wells containing cells and cultivated for 3 hours, followed by centrifugation at 1500 rpm for 20 minutes. After the supernatant was removed, 100 μL of DMSO was added into each of the wells with uniform shaking for 15 minutes, followed by subjecting to enzyme immunoassay analysis. The absorbance of each of the well s containing cells at 490 nm was recorded, thereby calculating the cell survival rate. The viability was calculated based on the following equation. The data is shown in FIG. 5. From FIG. 5 it is revealed that, even when the chondroitin sulfate-polycaprolactone copolymer is up to 1 mg/mL, the cell viability remains larger than 80%, which means the chondroitin sulfate-polycaprolactone copolymer has no significant cytotoxicity to CRL-5802 cells.

$$Cell viability ( \% ) = \frac{OD 490 ( experimental group )}{OD 490 ( control group )} \times 100 \%$$

(b) Cytotoxicity test of the medical composition against lung cancer cell CRL-5802:

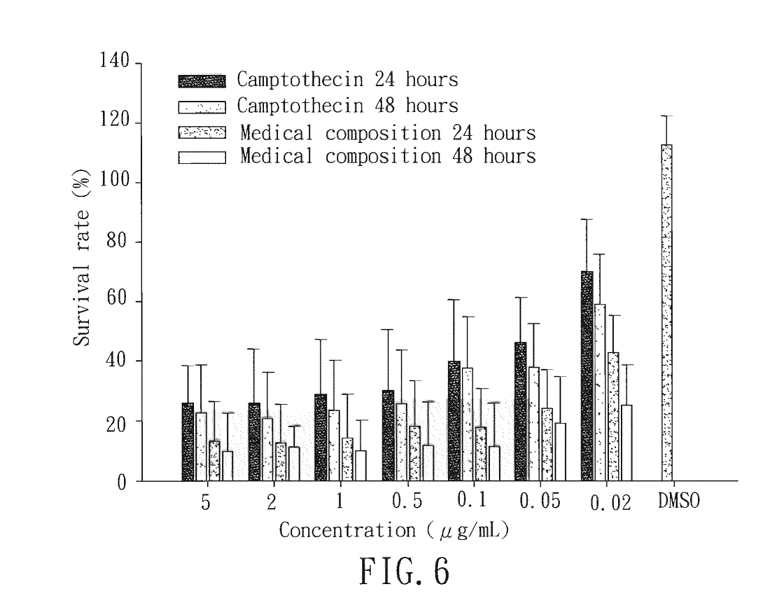

5000 cells/well of CRL-5802 were placed in a 96-well plate containing 100 μL of DMEM, followed by cultivation for 24 hours at 37° C. and 5% CO₂. The cell culture medium was periodically replaced during the cultivation period. Thereafter, the medical composition described in the section of “Preparation of medical composition” was mixed with DMEM to prepare an original solution having a concentration of 250 μg/mL (the concentration of camptothecin was 10 μg/mL). The original solution was diluted with DMEM to obtain six diluted solutions such that the concentrations of camptothecin in the six diluted solutions were respectively 4 μg/mL, 2 μg/mL, 1 μg/mL, 0.2 μg/mL, 0.1 μg/mL and 0.04 μg/mL. The original solution and the six diluted solutions were added into the respective wells containing cell s such that the final concentrations of camptothecin were adjusted to 5 μg/mL, 2 μg/mL, 1 μg/mL, 0.5 μg/mL, 0.1 μg/mL, 0.05 μg/mL, and 0.02 μg/mL respectively (experimental groups). The cells in the control group were cultured with DMEM without addition of the medical composition. The cells were further cultivated for 24 hours. Thereafter, the medium was removed and 100 μL of phosphate buffered saline (PBS) was then added into each of the wells containing cells. The PBS was then removed, followed by adding 100 μL of fresh medium. The cells were further cultivated for 24 hours or 48 hours. After that, 50 μL of MTT reagent was added into each of the well s containing cells and cultivated for 3 hours, followed by centrifugation at 1500 rpm for 20 minutes. After the supernatant was removed, 100 μL of DMSO was added into each of the wells containing cells with uniform shaking for 15 minutes, followed by subjecting to a measurement with the use of enzyme immunoassay analysis. The absorbance of each of the wells containing cells at 490 nm was recorded, thereby calculating the cell survival rate based on the aforesaid equation. The data is shown in FIG. 6.

(c) Cytotoxicity test of camptothecin against lung cancer cell CRL-5802:

The steps for determination of cytotoxicity of camptothecin against CRL-5802 were the same as those set forth in section 2(b). In this test, camptothecin and DMSO were mixed to prepare an original solution having a concentration of 2 mg/mL. The original solution was diluted with DMEM such that the final concentrations of camptothecin were adjusted to 10 μg/mL, 4 μg/mL, 2 μg/mL, 1 μg/mL, 0.2 μg/mL, 0.1 μg/mL and 0.04 μg/mL. Subsequently, the seven diluted solutions were added into the respective wells containing cells such that the concentrations of the solutions were changed respectively to 5 μg/mL, 2 μg/mL, 1 μg/mL, 0.5 μg/mL, 0.1 μg/mL, 0.05 μg/mL and 0.02 μg/mL. Since camptothecin cannot be dissolved in DMEM but can dissolved in DMEM containing DMSO, the cells in the DMSO group were cultured with DMEM and DMSO to determine the cytotoxicity effect of DMSO on the CRL-5802 cells. The results for the cytotoxicity test of camptothecin on CRL-5820 are shown in FIG. 6.

Based on the results shown in FIG. 6, it indicates that the medical composition of the present invention has an improved ability to kill CRL-5802 cells as compared to the case in which only camptothecin is used. For example, in the experimental groups that the camptothecin concentration is 1 μg/mL and the treatment time is 24 hours, the cell survival rate for the medical composition is about 15%, while the cell survival rate for camptothecin is 30%. In addition, the medical composition of the present invention has a significant killing ability to the CRL-5802 cells as time increases, which might indicate that the chondroitin sulfate-polycaprolactone copolymer of the present invention can slowly release the active ingredients to achieve an improved killing effect. FIG. 6 also shows that the DMSO has no cytotoxicity against CRL-5802 cells.

3. Cellular Uptake Ability

A cover glass having a diameter of 18 mm was immersed in 0.1 N HCl solution for one day, followed by washing with water and wiping using a clean tissue. The clean cover glass was immersed in 75% ethanol. Thereafter, the cover glass was removed from the ethanol by sterilized tweezers and was heated using a Bunsen burner to evaporate the residual ethanol on the cover glass.

The cover glass was placed in a well of a 12-well plate and 1×10³ cells/well of CRL-5802 were seeded on the cover glass and cultivated in DMEM containing 10% fetal bovine serum (FBS) for 24 hours. Thereafter, the chondroitin sulfate-polycaprolactone copolymer enclosed with 4% Nile red was added into the well, followed by cultivation for 30 minutes. Subsequently, the medium in the well was removed, and the cells were washed with PBS five times. Thereafter, 1 mL of 3.7% paraformaldehyde was added into each well containing the cover glass and cultivated for 30 minutes. Paraformaldehyde was then removed and the cells were washed with PBS five times.

Subsequently, 1 mL/well of 0.1% Triton X-100 (Manufacturer: Fluka) was added into the well, followed by cultivation for 5 minutes and then removal of Triton X-100. The cells were washed with PBS five times. Next, 0.5 μg/mL of 4′,6-diamidino-2-phenylindole (DAPI) was added into the well (0.5 mL/well) to stain the cell nuclei, followed by cultivation for 5 minutes, removal of DAPI, and washing with PBS five times. A drop of fluorescent mounting medium (Manufacturer: DakoCytomation) was added on a slide glass at the center thereof. The treated cover glass was removed from the plate and placed on the slide glass. Specifically, a side of the cover glass coated with the cells was faced and attached to the slide glass. The edges of the cover glass were sealed with a nail-polish oil and allowed to dry at room temperature.

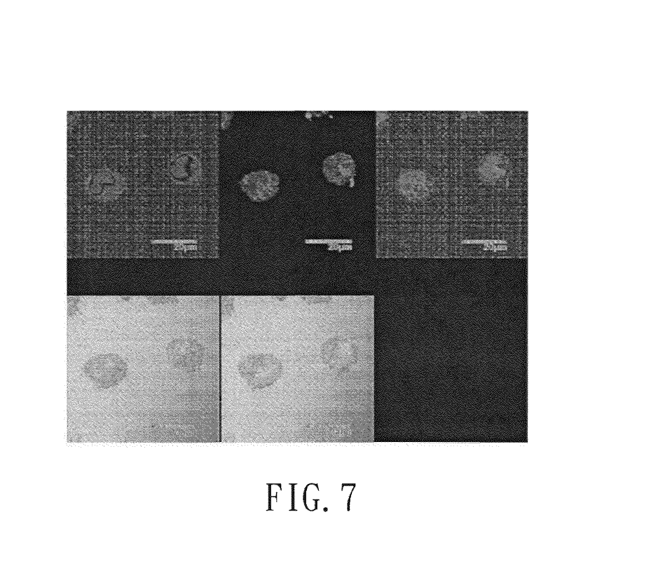

The cells were observed under a laser scanning confocal microscope (Olympus, Model No.: FV500). Results of the observation are shown in FIG. 7. From FIG. 7, it is found that the nano-micelle carrier is internalized into the CRL-5802 cells, which means the nano-micelle carrier of the present invention has an improved biocompatibility with the CRL-5802 cells.

4. Drug Releasability

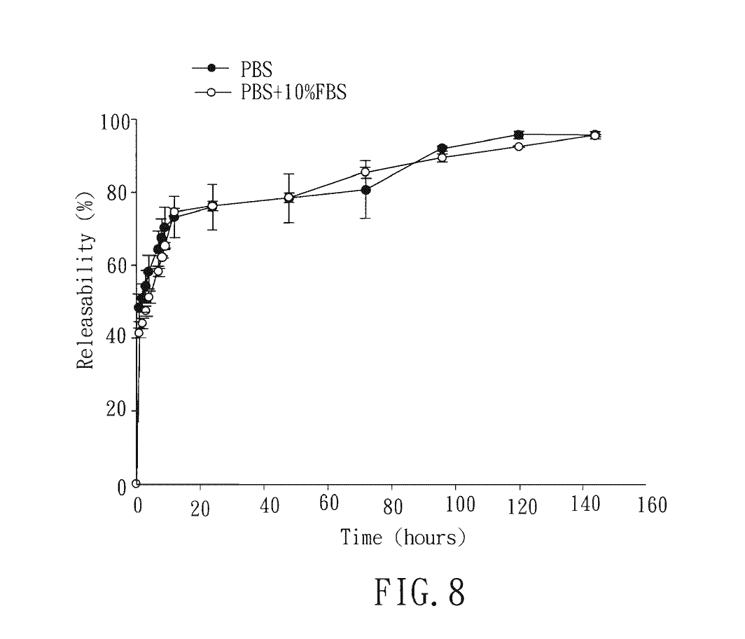

One mg of the medical composition obtained in the section of “Preparation of medical composition” was added to PBS with or without containing 10% FBS (pH=7.4) and cultivated at a 37° C. incubator. Supernatant were collected at different time points by centrifugation at 12,000 rpm for 5 minutes, and absorbance at 368 nm for each of the supernatants was measured using an ultraviolet spectrophotometer. The time points were 1, 2, 3, 4, 7, 8, 9, 12, 24, 48, 72, 96, 120 and 144 hours. The measurement was repeated three times and the result was represented by an average value. The absorbance values were collected and the released concentrations of camptothecin in the presence and in the absence of FBS were calculated. The graph showing time versus the released concentration of camptothecin is shown in FIG. 8. From FIG. 8, it is revealed that the medical composition may effectively release camptothecin as time increases, and may release camptothecin up to 75% within 20 hours, which means the chondroitin sulfate-polycaprolactone copolymer of the present invention has an improved ability to release the active ingredients. Moreover, in the presence of serum (FBS), the medical composition of the present invention still can release camptothecin up to 75% and effectively release camptothecin as time increases, which indicates that the release of camptothecin from the medical composition is not affected by serum.

5. Carriability of medical composition

One mg of the medical composition obtained in the section of “Preparation of medical composition” was dissolved in 2 mL of DMSO containing 4 μL of TFA at 60° C., followed by measuring the absorbance at a wavelength of 368 nm using an ultraviolet spectrophotometer to calculate the content of the camptothecin. The carriability of the camptothecin can be further calculated by the aforesaid equation (1).

The carriability of the medical composition of the present invention is 3.9%±0.15 for camptothecin. The doxorubicin was also used as an active ingredient in the carriability test, and the result is about 4.7%.

6. Enclosed Percentage of Medical Composition

One mg of the medical composition obtained in the section of “Preparation of medical composition” was dissolved in 2 mL of DMSO containing 4 μL of TFA at 60° C., followed by measuring the absorbance at a wavelength of 368 nm using an ultraviolet spectrophotometer to calculate the content of the camptothecin of the medical composition. The enclosed percentage of the camptothecin can be further calculated by the following equation:

$$Enclosed percentage ( \% ) = \frac{{W}_{c}}{{W}_{tc}} \times 100 \% \qquad ( 2 )$$

where W_{c} is the total amount of the enclosed camptothecin; and W_{tc} is the total amount of the camptothecin used to prepare the medical composition.
In the equation (2), the total amount of the camptothecin used to prepare the medical composition can be found in the section of “Preparation of medical composition”.

To sum up, by means of the method for preparing a chondroitin sulfate-polycaprolactone copolymer of the present invention using an atom transfer radical polymerization reaction, the grafting ratio and the reaction selectivity can be effectively increased, thereby reducing the generation of by-products so as to avoid complicated purification steps. In addition, since the chondroitin sulfate-polycaprolactone copolymer obtained by the abovementioned method has a lower critical micelle concentration and an improved biocompatibility, when it is used as a carrier, the micelles can be easily formed and the content of the active ingredient delivered to and internalized by the target cells can be increased.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A method for preparing a chondroitin sulfate-polycaprolactone copolymer, comprising subjecting a chondroitin sulfate component and a polycaprolactone polymer to an atom transfer radical polymerization reaction in the presence of a catalyst.

2. The method of claim 1, wherein the chondroitin sulfate component includes a modified chondroitin sulfate modified with a double-bond compound, and the polycaprolactone polymer is represented by the following formula (I) and has a weight average molecular weight ranging from 2000 to 10000, 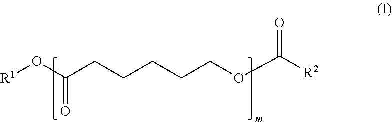
wherein, in formula (I), R¹ is a C₁-C₈ straight or branched alkyl group, an aromatic group, or 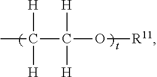
R¹¹ being H or methyl, t being an integer ranging from 45 to 225;
R² is 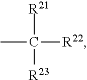
R²¹, R²², and R²³ being independently H, methyl, or halogen atom, with the proviso that at least one of R²¹, R²², and R²³ is halogen atom, and
m is an integer ranging from 18 to 88.

3. The method of claim 2, wherein the double-bond compound is selected from the group consisting of acrylic acid, acrylic anhydride, acryol chloride, methacrylic acid, methacrylic anhydride, methacryol chloride, and methyl methacrylate.

4. The method of claim 1, wherein the weight ratio of the chondroitin sulfate component to the polycaprolactone polymer ranges from 0.1 to 0.9.

5. The method of claim 2, wherein the modified chondroitin sulfate is obtained by subjecting chondroitin sulfate and the double-bond compound to a polymerization reaction.

6. The method of claim 5, wherein the molar ratio of the chondroitin sulfate to the double-bond compound ranges from 0.05 to 0.8.

7. The method of claim 2, wherein, in formula (I), R¹ is a C₁-C₈ straight or branched alkyl group, a benzyl group, 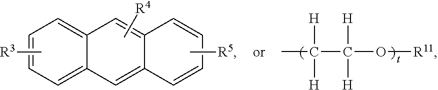
wherein R¹¹ is H or methyl, R³, R⁴, and R⁵ are independently H or an alkylene group, with the proviso that at least one of R³, R⁴, and R⁵ is an alkylene group.

8. The method of claim 2, wherein, in formula (I), R² is 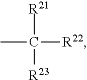
R²¹, R²², and R²³ being independently H, methyl, Cl, or Br, with the proviso that at least one of R²¹,R²², and R²³ is Cl or Br.

9. A chondroitin sulfate-polycaprolactone copolymer, which is prepared according to the method of claim 1.

10. A nano-micelle carrier obtained by subjecting the chondroitin sulfate-polycaprolactone copolymer of claim 9 to a dialysis treatment.

11. A medical composition comprising the chondroitin sulfate-polycaprolactone copolymer of claim 9 and an active ingredient.

12. A medical composition comprising the nano-micelle carrier of claim 10 and an active ingredient.
